(19) United States
(12) Patent Application Publication
(10) Pub. No.: US 2005/0266021 A1
(43) Pub. Date: Dec. 1, 2005

(54) Powder compositions

(75) Inventors: Yuh-Fun Maa, Fremont, CA (US); Lu Zhao, Fremont, CA (US); Steven J. Prestrelski, Fremont, CA (US)

Correspondence Address: Chiron Corporation;Intellectual Property - R440, P.O. Box 8097, Emeryville, CA, 94662-8097, US

(73) Assignee: Powderject Vaccines, Inc.

(21) Appl. No.: 11/041,875

(22) Filed: Jan. 25, 2005

Related U.S. Application Data
(63) Continuation of application No. 09/877,726, filed on Jun. 8, 2001, now abandoned.
(60) Provisional application No. 60/210,581, filed on Jun. 8, 2000.

(51) Int. Cl. A61K 39/12; A61K 39/02

(52) U.S. Cl. 424/204.1; 424/234.1

(57) ABSTRACT

A gel-forming free-flowing powder suitable for use as a vaccine is prepared by spray-drying or spray freeze-drying an aqueous suspension that contains an antigen adsorbed to an aluminum salt or calcium salt adjuvant, a saccharide, an amino acid or a salt thereof, and a colloidal substance. Powder for vaccine purposes are also prepared by spray freeze-drying an aqueous suspension of such an adjuvant having an antigen adsorbed therein. Processes for forming these powder compositions are also described, as well as methods of using the compositions in a vaccination procedure.

### CROSS-REFERENCE TO RELATED APPLICATION

This application is related to U.S. provisional application Ser. No. 60/210,581, filed 8 Jun. 2000, from which priority is claimed pursuant to 35 U.S.C. §119(e)(1) and which application is incorporated herein by reference in its entirety.

### FIELD OF THE INVENTION

The invention relates to vaccine compositions. More specifically, the invention relates to vaccine compositions suitable for transdermal particle delivery from a needleless syringe system.

### BACKGROUND TO THE INVENTION

The ability to deliver pharmaceutical agents into and through skin surfaces (transdermal delivery) provides many advantages over oral or parenteral delivery techniques. In particular, transdermal delivery provides a safe, convenient and noninvasive alternative to traditional administration systems, conveniently avoiding the major problems associated with oral delivery (e.g. variable rates of absorption and metabolism, gastrointestinal irritation and/or bitter or unpleasant drug tastes) or parenteral delivery (e.g. needle pain, the risk of introducing infection to treated individuals, the risk of contamination or infection of health care workers caused by accidental needle-sticks and the disposal of used needles).

However, despite its clear advantages, transdermal delivery presents a number of its own inherent logistical problems. Passive delivery through intact skin necessarily entails the transport of molecules through a number of structurally different tissues, including the stratum corneum, the viable epidermis, the papillary dermis and the capillary walls in order for the drug to gain entry into the blood or lymph system. Transdermal delivery systems must therefore be able to overcome the various resistances presented by each type of tissue.

In light of the above, a number of alternatives to passive transdermal delivery have been developed. These alternatives include the use of skin penetration enhancing agents, or “permeation enhancers,” to increase skin permeability, as well as non-chemical modes such as the use of iontophoresis, electroporation or ultrasound. However, these alternative techniques often give rise to their own unique side effects such as skin irritation or sensitization. Thus, the spectrum of agents that can be safely and effectively administered using traditional transdermal delivery methods has remained limited.

More recently, a novel transdermal drug delivery system that entails the use of a needleless syringe to fire powders (i.e., solid drug-containing particles) in controlled doses into and through intact skin has been described. In particular, commonly owned U.S. Pat. No. 5,630,796 to Bellhouse et al. describes a needleless syringe that delivers pharmaceutical particles entrained in a supersonic gas flow. The needleless syringe is used for transdermal delivery of powdered drug compounds and compositions, for delivery of genetic material into living cells (e.g., gene therapy) and for the delivery of biopharmaceuticals to skin, muscle, blood or lymph. The needleless syringe can also be used in conjunction with surgery to deliver drugs and biologics to organ surfaces, solid tumors and/or to surgical cavities (e.g., tumor beds or cavities after tumor resection). In theory, practically any pharmaceutical agent that can be prepared in a substantially solid, particulate form can be safely and easily delivered using such devices.

One area of the pharmaceuticals field which is of particular interest for delivery via this new system is that of vaccine compositions. Suitable vaccines include those comprising an antigen adsorbed into a salt adjuvant. Such compositions are known in the art (see for example U.S. Pat. No. 5,902,565) and are advantageous since the adjuvant enhances the immunogenicity of the vaccine.

However, the storage and transportation of adjuvant vaccines is problematic. Commercial vaccine compositions containing salt adjuvants cannot be frozen without causing damage to the vaccine. Further, one of the common storage techniques currently used for vaccines, freeze-drying, is also unavailable for salt adjuvant containing compositions. Previous research has demonstrated that freeze-drying causes the collapse of the gel structure of the vaccine composition, resulting in aggregation and precipitation of the adjuvant salt on resuspension in water (Warren et al, 1986, Annu. Rev. Immunol. 4: pages 369-388; Alving et al, Ann. N.Y. Acad. Sci. 690: pages 265-275). This is believed to be due to crystallisation of the water contained in the composition into large crystals on freezing and hence the concentration of the solute into specific regions, known as freeze concentrate regions. In the freeze concentrate regions, adjuvant salt particles are brought into close proximity and repulsive forces are overcome, thereby resulting in coagulation. Once the salt has coagulated, the original suspension cannot be reproduced. This effect has been found to significantly reduce the immunogenicity of the vaccine, one report demonstrating a complete loss in immunogenicity of a freeze-dried alum-adsorbed hepatitis B surface antigen (HBsAg) after storage at 4° C. for two years (Diminsky et al, Vaccine, 18: pages 3-17).

An alternative method for storing adjuvant vaccine compositions is therefore required, which addresses the problems of aggregation associated with freeze-drying and which provides maximum retention of immunogenicity. Prolonged storage of vaccines is essential, both for use with the novel transdermal drug delivery systems mentioned above and also for use with conventional vaccination techniques. The provision of an effective alternative to freeze-drying is therefore of considerable commercial importance. It is also desired that the vaccine be produced in a form suitable for needleless injection. Needleless injection requires the vaccine composition to be in powder form, each particle having a suitable size and strength for transdermal delivery and being capable of forming a gel on resuspension.

Alternatives to conventional freeze-drying techniques that have previously been reported include the incorporation of additives in the vaccine composition to improve the stability of an alum adjuvant. U.S. Pat. No. 4,578,270 describes the addition of large amounts of both dextran and protein in order to achieve partial retention of the aluminum gel structure. This large addition of protein could however act to displace vaccine antigens from the aluminum gel and in addition would, in most cases, be immunogenic and as a result tend to swamp the immune response to the vaccine antigen.

EP-B-0130619 is also concerned with the addition of stabilisers to lyophilised, or freeze-dried, vaccine preparations. Lyophilised preparations of a hepatitis B vaccine comprising an inactivated purified hepatitis B virus surface antigen absorbed an aluminum gel and stabiliser are described. The stabiliser is composed of at least one amino acid or salt thereof, at least one saccharide and at least one colloidal substance. Very low concentrations of aluminum salt adjuvant are used, typically less than 0.1% by weight. However, this document relates only to the hepatitis B vaccine and does not disclose a generic process, which is non-immunogen-specific.

Spray-dried vaccine preparations comprising an immunogen adsorbed into an aluminum salt are disclosed in U.S. Pat. No. 5,902,565. Immediate-release preparations are described which are prepared by spray-drying an aqueous suspension of aluminum salt-adsorbed immunogen. In the only Example, Example 1, in which such information is given, the resultant microspheres had a size range around 3 μm in diameter. According to U.S. Pat. No. 5,902,565 the gel-forming nature of aluminum gels is completely retained during spray-drying even in the absence of any other materials which could exert a stabilising effect (apart from minimal quantities of vaccine antigen, typically 1 to 10 μg/ml). Addition of water to the spray-dried powder was said to result in the instant formation of a typical gel, with sedimentation properties similar to the starting material.

### SUMMARY OF THE INVENTION

We investigated whether a gel-forming spray-dried powder of an aluminum salt could indeed be formed as described in U.S. Pat. No. 5,902,565. We found that spray drying a suspension of aluminum hydroxide or aluminum phosphate in water caused submicron particles of the aluminum salt to aggregate to larger particles in the resulting spray-dried powder. Upon reconstitution of this powder in water, these larger particles did not disintegrate into small particles. A gel suspension did not form. Rather, the aggregated particles of aluminum hydroxide or aluminum phosphate sedimented and precipitated out of the suspension.

Further experiments were carried out. We found that a suitable powder could be formed by spray-drying when an aluminum salt was utilised with a specific combination of other agents. Additionally, the aluminum salt and other agents needed to be used in specific proportions. We found too that the particular drying method used has a significant effect on the degree of coagulation of the adjuvant salt. These investigations led to the finding that a powder suitable for needleless injection, and which substantially retained its gel structure on reconstitution in water, was obtainable by spray freeze-drying an alum adjuvant vaccine composition.

The spray freeze-drying method involves atomizing the suspended vaccine composition into liquid nitrogen. This process has two important effects: firstly, the liquid nitrogen acts as a heat transfer agent and provides rapid freezing of the suspension; and secondly, the atomisation reduces the volume of each droplet to be frozen, further increasing the freezing rate. This combined effect causes extremely rapid freezing of very small droplets of suspension and leads to the formation of smaller ice crystals in the solid. The freeze concentrate regions which form during a standard freeze-drying technique are therefore significantly reduced in size. The rapid freezing of the particles, and their small size leads to powders having little or no aggregated adjuvant.

The present invention therefore provides simple, yet effective techniques that generate salt adjuvant-containing vaccine compositions in a powder form which is suitable for long-term storage. The vaccine compositions of the invention show substantially no aggregation on reconstitution and therefore immunogenicity is substantially retained. The compositions also have well-defined particle size, density and mechanical properties which collectively are suitable for powders for transdermal delivery from a needleless syringe.

The invention has the further, significant advantage that it is suitable for use with a wide range of vaccine compositions and may well also be applicable to other pharmaceutical compositions, in particular where similar aggregation problems are encountered. As yet, the spray freeze-drying technique has been found to be entirely formulation independent within the field of adjuvant vaccine compositions.

Accordingly, the present invention provides a gel-forming free-flowing powder suitable for use as a vaccine, said powder being obtainable by spray-drying or spray freeze-drying an aqueous suspension comprising:
(a) from 0.1 to 0.95% by weight of an aluminum salt or calcium salt adjuvant having an antigen adsorbed thereon;
(b) from 0.5 to 6% by weight of saccharide;
(c) from 0.1 to 2% by weight of an amino acid or salt thereof; and
(d) from 0.02 to 1% by weight of a colloidal substance.

Free-flowing powder compositions suitable for vaccine use can thus be produced. The compositions have well-defined particle size, density and mechanical properties which collectively are suitable for powders for transdermal delivery from a needleless syringe. The invention further provides:
a process for the preparation of a gel-forming free-flowing powder suitable for use as a vaccine, which process comprises spray-drying or spray freeze-drying an aqueous suspension comprising:
(a) from 0.1 to 0.95% by weight of an aluminum salt or calcium salt adjuvant having an antigen adsorbed therein;
(b) from 0.5 to 6% by weight of a saccharide;
(c) from 0.1 to 2% by weight of an amino acid or salt thereof; and
(d) from 0.02 to 1% by weight of a colloidal substance;
a dosage receptacle for a needleless syringe, said receptacle containing an effective amount of a powder of the invention;
a needleless syringe which is loaded with a powder of the invention;
a vaccine composition comprising a pharmaceutically acceptable carrier or diluent and a powder of the invention;
a method of vaccinating a subject, which method comprises administering to the said subject an effective amount of a powder of the invention; and
a gel-forming free-flowing powder suitable for use as a vaccine, which powder comprises:
(i) from 5 to 60% by weight of an aluminum salt or calcium salt adjuvant having an antigen adsorbed thereon;
(ii) from 25 to 90% by weight of a saccharide;
(iii) from 4.5 to 40% by weight of an amino acid or salt thereof; and
(iv) from 0.5 to 10% by weight of a colloidal substance.

Additionally, the present invention provides a powder suitable for use as a vaccine, said powder being obtainable by spray freeze-drying an aqueous suspension comprising an aluminum salt or calcium salt adjuvant having an antigen adsorbed therein.

The invention further provides:
a process for the preparation of a powder suitable for use as a vaccine, which process comprises spray freeze-drying an aqueous suspension comprising an aluminum salt or calcium salt adjuvant having an antigen adsorbed therein;
a dosage receptacle for a needleless syringe, said receptacle containing an effective amount of such a spray freeze-dried powder of the invention;
a needleless syringe which is loaded with this spray freeze-dried powder of the invention;
a vaccine composition comprising a pharmaceutically acceptable carrier or diluent and the spray freeze-dried powder of the invention; and
a method of vaccinating a subject, which method comprises administering to the said subject an effective amount of the spray freeze-dried powder of the invention.

### BRIEF DESCRIPTION OF THE DRAWINGS

FIG. 1 shows the particle size distribution of an HBsAg adsorbed alum gel (i) before drying and (ii) after drying using a spray freeze-drying technique followed by reconstitution in water.

FIG. 2 shows the particle size distribution of a second HBsAg adsorbed alum gel before drying and after drying via a conventional freeze drying method.

FIG. 3 illustrates the results of an immunogenicity study using mice injected with HBsAg absorbed alum vaccine which had been dried by either spray freeze-drying (SFD) according to present invention, or using freeze-drying (FD). The FD powders were sieved into different size fractions and tested for immunogenicity. Two SFD formulations, varying in alum contact, were tested.

FIG. 4 illustrates the immunogenicity of three different spray freeze-dried powders in mice immunized by either intramuscular injection using a needle or epidermal powder immunization using a powder delivery device.

FIG. 5 illustrates the immunogenicity of spray freeze-dried diphtheria-tetanus toxoid vaccine in guinea pigs. Spray freeze-dried powders of 20-38 μm and 38-53 μm in diameter were administered as a powder to the abdominal skin using a powder delivery device.

### DETAILED DESCRIPTION OF THE PREFERRED EMBODIMENTS

Before describing the present invention in detail, it is to be understood that this invention is not limited to particularly exemplified compositions or process parameters as such may, of course, vary. It is also to be understood that the terminology used herein is for the purpose of describing particular embodiments of the invention only, and is not intended to be limiting.

All publications, patents and patent applications cited herein, whether supra or infra, are hereby incorporated by reference in their entirety.

It must be noted that, as used in this specification and the appended claims, the singular forms “a”, “an” and “the” include plural referents unless the content clearly dictates otherwise. Thus, for example, reference to “a particle” includes a mixture of two or more such particles, reference to “an excipient” includes mixtures of two or more such excipients, and the like.

A. Definitions

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

In describing the present invention, the following terms will be employed, and are intended to be defined as indicated below. By “antigen” is meant a molecule which contains one or more epitopes that will stimulate a host's immune system to make a cellular antigen-specific immune response or a humoral antibody response. Thus, antigens include polypeptides including antigenic protein fragments, oligosaccharides, polysaccharides and the like. Furthermore, the antigen can be derived from any known virus, bacterium, parasite, plant, protozoan or fungus, and can be a whole organism. The term also includes tumor antigens. Similarly, an oligonucleotide or polynucleotide which expresses an antigen, such as in DNA immunization applications, is also included in the definition of an antigen. Synthetic antigens are also included, for example polyepitopes, flanking epitopes and other recombinant or synthetically derived antigens (Bergmann et al (1993) Eur. J. Immunol. 23:2777-2781; Bergmann et al. (1996) J. Immunol. 157:3242-3249; Suhrbier, A. (1997) Immunol. and Cell Biol. 75:402-408; Gardner et al. (1998) 12^{th} World AIDS Conference, Geneva, Switzerland, Jun. 28-Jul. 3, 1998).

The aduvants having antigen adsorbed thereon of the present invention, alone or in combination, are typically combined with one or more added materials such as carriers, vehicles, and/or excipients. “Carriers,” “vehicles” and “excipients” generally refer to substantially inert materials which are nontoxic and do not interact with other components of the composition in a deleterious manner. These materials can be used to increase the amount of solids in particulate pharmaceutical compositions. Examples of suitable carriers include water, silicone, gelatin, waxes, and like materials. Examples of normally employed “excipients,” include pharmaceutical grades of carbohydrates including monosaccharides, disaccharides, cyclodextrans, and polysaccharides (e.g., dextrose, sucrose, lactose, trehalose, raffinose, mannitol, sorbitol, inositol, dextrans, and maltodextrans); starch; cellulose; salts (e.g. sodium or calcium phosphates, calcium sulfate, magnesium sulfate); citric acid; tartaric acid; glycine; high molecular weight polyethylene glycols (PEG); Pluronics; surfactants; and combinations thereof. Generally, when carriers and/or excipients are used, they are used in amounts ranging from about 0.1 to 99 wt % of the pharmaceutical composition.

The term “powder” as used herein refers to a composition that consists of substantially solid particles that can be delivered transdermally using a needleless syringe device. The particles that make up the powder can be characterized on the basis of a number of parameters including, but not limited to, average particle size, average particle density, particle morphology (e.g. particle aerodynamic shape and particle surface characteristics) and particle penetration energy (P.E.).

The average particle size of the powders according to the present invention can vary widely and is generally from 0.1 to 250 μm, for example from 10 to 100 μm and more typically from 20 to 70 μm. The average particle size of the powder can be measured as a mass mean aerodynamic diameter (MMAD) using conventional techniques such as microscopic techniques (where particles are sized directly and individually rather than grouped statistically), absorption of gases, permeability or time of flight. If desired, automatic particle-size counters can be used (e.g. Aerosizer Counter, Coulter Counter, HIAC Counter, or Gelman Automatic Particle Counter) to ascertain the average particle size.

Actual particle density or “absolute density” can be readily ascertained using known quantification techniques such as helium pycnometry and the like. Alternatively, envelope (“tap”) density measurements can be used to assess the density of a powder according to the invention. The envelope density of a powder of the invention is generally from 0.1 to 25 g/cm³, preferably from 0.8 to 1.5 g/cm³.

Envelope density information is particularly useful in characterizing the density of objects of irregular size and shape. Envelope density is the mass of an object divided by its volume, where the volume includes that of its pores and small cavities but excludes interstitial space. A number of methods of determining envelope density are known in the art, including wax immersion, mercury displacement, water absorption and apparent specific gravity techniques. A number of suitable devices are also available for determining envelope density, for example, the GeoPyc™ Model 1360, available from the Micromeritics Instrument Corp. The difference between the absolute density and envelope density of a sample pharmaceutical composition provides information about the sample's percentage total porosity and specific pore volume.

Particle morphology, particularly the aerodynamic shape of a particle, can be readily assessed using standard light microscopy. It is preferred that the particles which make up the instant powders have a substantially spherical or at least substantially elliptical aerodynamic shape. It is also preferred that the particles have an axis ratio of 3 or less to avoid the presence of rod- or needle-shaped particles. These same microscopic techniques can also be used to assess the particle surface characteristics, e.g. the amount and extent of surface voids or degree of porosity.

Particle penetration energies can be ascertained using a number of conventional techniques, for example a metallized film P.E. test. A metallized film material (e.g. a 125 μm polyester film having a 350 Å layer of aluminum deposited on a single side) is used as a substrate into which the powder is fired from a needleless syringe (e.g. the needleless syringe described in U.S. Pat. No. 5,630,796 to Bellhouse et al) at an initial velocity of about 100 to 3000 m/sec. The metallized film is placed, with the metal-coated side facing upwards, on a suitable surface.

A needleless syringe loaded with a powder is placed with its spacer contacting the film, and then fired. Residual powder is removed from the metallized film surface using a suitable solvent. Penetration energy is then assessed using a BioRad Model GS-700 imaging densitometer to scan the metallized film, and a personal computer with a SCSI interface and loaded with MultiAnalyst software (BioRad) and Matlab software (Release 5.1, The MathWorks, Inc.) is used to assess the densitometer reading. A program is used to process the densitometer scans made using either the transmittance or reflectance method of the densitometer. The penetration energy of the spray-coated powders should be equivalent to, or better than that of reprocessed mannitol particles of the same size (mannitol particles that are freeze-dried, compressed, ground and sieved according to the methods of commonly owned International Publication No. WO 97/48485, incorporated herein by reference).

The term “subject” refers to any member of the subphylum cordata including, without limitation, humans and other primates including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The methods described herein are intended for use in any of the above vertebrate species, since the immune systems of all of these vertebrates operate similarly.

The term “transdermal delivery” includes both transdermal (“percutaneous”) and transmucosal routes of administration, i.e. delivery by passage through the skin or mucosal tissue. See, e.g., Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermal Delivery of Drugs, Vols. 1-3, Kydonieus and Berner (eds.), CRC Press, (1987).

B. General Methods

The invention is concerned with gel-forming free-flowing powders suitable for use as vaccines. The powders are suitable for transdermal administration from a needleless syringe delivery system. As such, the particles which make up the powdered composition must have sufficient physical strength to withstand sudden acceleration to several times the speed of sound and the impact with, and passage through, the skin and tissue. The particles are formed by spray-drying or spray freeze-drying an aqueous suspension comprising or, in some embodiments, consisting essentially of:
(a) from 0.1 to 0.95% by weight of an aluminum salt or calcium salt adjuvant having an antigen adsorbed therein;
(b) from 0.5 to 6% by weight of a saccharide;
(c) from 0.1 to 2% by weight of an amino acid or salt thereof; and
(d) from 0.02 to 1% by weight of a colloidal substance.

The aqueous suspension contains, as component (a), less than 1% by weight of the adjuvant having antigen adsorbed thereon. Preferably, the suspension contains from 0.2 or 0.3 to 0.6 or 0.75% by weight, preferably from 0.2 to 0.4% by weight, of the adjuvant onto which antigen is adsorbed. The aluminum salt adjuvant is generally aluminum hydroxide or aluminum phosphate. Alternatively, the adjuvant may be aluminum sulfate or calcium phosphate.

Any suitable antigen as defined herein may be employed. The antigen may be a viral antigen. The antigen may therefore be derived from members of the families Picornaviridae (e.g. polioviruses, etc.); Caliciviridae; Togaviridae (e.g. rubella virus, dengue virus, etc.); Flaviviridae; Coronaviridae; Reoviridae; Birnaviridae; Rhabodoviridae (e.g. rabies virus, etc.); Filoviridae; Paramyxoviridae (e.g. mumps virus, measles virus, respiratory syncytial virus, etc.); Orthomyxoviridae (e.g. influenza virus types A, B and C, etc.); Bunyaviridae; Arenaviridae; Retroviradae (e.g. HTLV-I; HTLV-II; HIV-1 and HIV-2); and simian immunodeficiency virus (SIV) among others.

Alternatively, viral antigens may be derived from papillomavirus (e.g. HPV); a herpesvirus; a hepatitis virus, e.g. hepatitis A virus (HAV), hepatitis B virus (HBV), hepatitis C(HCV), the delta hepatitis virus (HDV), hepatitis E virus (HEV) or hepatitis G virus (HGV); and the tick-borne encephalitis viruses. See, e.g. Virology, 3rd Edition (W. K. Joklik ed. 1988); Fundamental Virology, 2nd Edition (B. N. Fields and D. M. Knipe, eds. 1991) for a description of these viruses.

Bacterial antigens for use in the invention can be derived from organisms that cause diphtheria, cholera, tuberculosis, tetanus, pertussis, meningitis and other pathogenic states, including, e.g., Meningococcus A, B and C, Hemophilus influenza type B (HIB), Helicobacter pylori, Vibrio cholerae, Escherichia coli, Campylobacter, Shigella, Salmonella, Streptococcus sp, and Staphylococcus sp. A combination of bacterial antigens may be provided, for example diphtheria, pertussis and tetanus antigens. Suitable pertussis antigens are pertussis toxin and/or filamentous haemagglutinin and/or pertactin, alternatively termed P69. An anti-parasitic antigen may be derived from organisms causing malaria and Lyme disease.

Antigens for use in the present invention can be produced using a variety of methods known to those of skill in the art. In particular, the antigens can be isolated directly from native sources, using standard purification techniques. Alternatively, whole killed, attenuated or inactivated bacteria, viruses, parasites or other microbes may be employed. Yet further, antigens can be produced recombinantly using known techniques. See, e.g., Sambrook, Fritsch & Maniatis, Molecular Cloning: A Laboratory Manual, Vols. I and II (D. N. Glover et. 1985).

Antigens for use herein may also be synthesised, based on described amino acid sequences, via chemical polymer syntheses such as solid phase peptide synthesis. Such methods are known to those of skill in the art. See, e.g. J. M. Stewart and J. D. Young, Solid Phase Peptide Synthesis, 2nd Ed., Pierce Chemical Co., Rockford, Ill. (1984) and G. Barany and R. B. Merrifield, The Peptides: Analysis, Synthesis, Biology, editors E. Gross and J. Meienhofer, Vol. 2, Academic Press, New York, (1980), pp. 3-254, for solid phase peptide synthesis techniques; and M. Bodansky, Principles of Peptide Synthesis, Springer-Verlag Berlin (1984) and E. Gross and J. Meienhofer, Eds., The Peptides: Analysis, Synthesis, Biology, supra, Vol. 1, for classical solution synthesis.

One or more saccharides may be present in the aqueous suspension as component (b). The saccharide content is typically 1.5 to 5% by weight, preferably 2 to 4% by weight. The saccharide may be a monosaccharide such as glucose, xylose, galactose, fructose, D-mannose or sorbose; a disaccharide such as lactose, maltose, saccharose, trehalose or sucrose; or a sugar alcohol such as mannitol, sorbitol, xylitol, glycerol, erythritol or arabitol.

One or more amino acids or amino acid salts is present in the aqueous suspension as component (c). Any physiologically acceptable amino acid salt may be employed. The salt may be an alkali or alkaline earth metal salt such as sodium, potassium or magnesium salt. The amino acid may be an acidic, neutral or basic amino acid. Suitable amino acids are glycine, alanine, glutamine, arginine, lysine and histidine. Monosodium glutamate is a suitable amino acid salt. The aqueous suspension generally contains from 0.5 to 1.5% by weight, more preferably from 0.75 to 1.25% by weight, of the amino acid and/or amino acid salt.

The colloidal substance (d) is a divided substance incapable of passing through a semi-permeable membrane, comprised of fine particles which, in suspension or solution, fail to settle out. Suitable colloidal substances are disclosed in EP-B-0130619. Component (d) may be selected from polysaccharides such as dextran or maltodextran; hydrogels such as gelatin or agarose; or proteins such as human serum albumin. The substance may have a molecular weight of 500 to 80,000 or higher, for example from 1000 or 2000 to 30,000 or from 5,000 to 25,000. Component (d) is generally present in the aqueous suspension in an amount of from 0.05 to 0.5% by weight, preferably from 0.07 to 0.3% by weight.

The adjuvant having antigen adsorbed thereon and the saccharide, amino acid or salt thereof and colloidal substance are suspended in water. The aqueous suspension is spray dried or spray freeze-dried. The spray-drying or spray freeze-drying conditions are selected to enable the desired particles to be produced. The air inlet temperature, air outlet temperature, feed rate of the aqueous suspension, air flow rate, etc. can thus be varied as desired. Any suitable spray-drier may be used. The nozzle size may vary as necessary. Particular spray freeze-drying conditions are described in more detail below.

A gel-forming free-flowing powder can thus be provided which is suitable for use as a vaccine. The proportions of the various components of the powder can be adjusting by adjusting the composition of the suspension that is spray-dried or spray freeze-dried. However, the powder typically comprises or, in some embodiments, consists essentially of:
(i) from 5 to 60%, for example from 7 to 50% such as from 10 to 30%, by weight of an aluminum salt or calcium salt adjuvant having an antigen adsorbed thereon;
(ii) from 25 to 90%, for example from 30 to 80% such as from 40 to 70%, by weight of a saccharride;
(iii) from 4.5 to 40%, for example from 7 to 30% such as from 10 to 20%, by weight of an amino acid or salt thereof; and
(iv) from 0.5 to 10%, for example from 0.8 to 6% such as from 1 to 3%, by weight of a colloidal substance.

The invention is concerned generally with powders suitable for use as vaccines that are formed by spray freeze-drying an aqueous suspension comprising an aluminum salt or calcium salt adjuvant having an antigen adsorbed therein. Such powders are suitable for transdermal administration from a needleless syringe delivery system. As such, the particles which make up the powdered composition must have sufficient physical strength to withstand sudden acceleration of up to several times the speed of sound and the impact with, and passage through, the skin and tissue.

Preferably, the aqueous suspension, prior to spray freeze-drying, contains less than 10% by weight, for instance less than 5% weight and preferably less than 3% by weight, of the salt adjuvant having antigen adsorbed thereon. The aqueous suspension typically contains at least 0.05% by weight, for instance at least 0.1% by weight or at least 0.6% by weight, of the adjuvant having antigen adsorbed thereon. More preferably, the suspension contains from 0.2 or 0.3 to 0.6%, 0.75% or 1% by weight, preferably from 0.2 to 0.4% by weight, of adjuvant onto which antigen is adsorbed. At concentrations above about 10% by weight of adjuvant salt, the aqueous suspension becomes highly viscous. This limits the ability to atomize the suspension.

It should be understood that the preferred upper limit of adjuvant concentration applies to the aqueous suspension prior to spray freeze-drying. The content of adjuvant salt having antigen adsorbed thereon may be as high as 50% by weight or more in the spray freeze-dried powders of the invention.

The adjuvant is generally an aluminum salt, for example aluminum hydroxide or aluminum phosphate. Alternatively, the adjuvant salt may be aluminum sulfate or calcium phosphate.

Again, any suitable antigen as defined herein may be employed. The antigen may be a viral antigen. The antigen may therefore be derived from members of the families Picornaviridae (e.g. polioviruses, etc.); Caliciviridae; Togaviridae (e.g. rubella virus, dengue virus, etc.); Flaviviridae; Coronaviridae; Reoviridae; Birnaviridae; Rhabodoviridae (e.g. rabies virus, etc.); Filoviridae; Paramyxoviridae (e.g. mumps virus, measles virus, respiratory syncytial virus, etc.); Orthomyxoviridae (e.g. influenza virus types A, B and C, etc.); Bunyaviridae; Arenaviridae; Retroviradae (e.g. HTLV-I; HTLV-II; HIV-1 and HIV-2); and simian immunodeficiency virus (SIV) among others.

Alternatively, viral antigens may be derived from papillomavirus (e.g. HPV); a herpesvirus; a hepatitis virus, e.g. hepatitis A virus (HAV), hepatitis B virus (HBV), hepatitis C(HCV), the delta hepatitis virus (HDV), hepatitis E virus (HEV) or hepatitis G virus (HGV); and the tick-borne encephalitis viruses. See, e.g. Virology, 3rd Edition (W. K. Joklik ed. 1988); Fundamental Virology, 2nd Edition (B. N. Fields and D. M. Knipe, eds. 1991) for a description of these viruses.

Bacterial antigens for use in the invention can be derived from organisms that cause diphtheria, cholera, tuberculosis, tetanus, pertussis, meningitis and other pathogenic states, including, e.g., Meningococcus A, B and C, Hemophilus influenza type B (HIB), Helicobacter pylori, Vibrio cholerae, Escherichia coli, Campylobacter, Shigella, Salmonella, Streptococcus sp, and Staphylococcus sp. A combination of bacterial antigens may be provided, for example diphtheria, pertussis and tetanus antigens. Suitable pertussis antigens are pertussis toxin and/or filamentous haemagglutinin and/or pertactin, alternatively termed P69. An anti-parasitic antigen may be derived from organisms causing malaria and Lyme disease.

Antigens for use in the present invention can be produced using a variety of methods known to those of skill in the art. In particular, the antigens can be isolated directly from native sources, using standard purification techniques. Alternatively, whole killed, attenuated or inactivated bacteria, viruses, parasites or other microbes may be employed. Yet further, antigens can be produced recombinantly using known techniques. See, e.g., Sambrook, Fritsch & Maniatis, Molecular Cloning: A Laboratory Manual, Vols. I and II (D. N. Glover et. 1985).

Antigens for use herein may also be synthesised, based on described amino acid sequences, via chemical polymer syntheses such as solid phase peptide synthesis. Such methods are known to those of skill in the art. See, e.g. J. M. Stewart and J. D. Young, Solid Phase Peptide Synthesis, 2nd Ed., Pierce Chemical Co., Rockford, Ill. (1984) and G. Barany and R. B. Merrifield, The Peptides: Analysis, Synthesis, Biology, editors E. Gross and J. Meienhofer, Vol. 2, Academic Press, New York, (1980), pp. 3-254, for solid phase peptide synthesis techniques; and M. Bodansky, Principles of Peptide Synthesis, Springer-Verlag Berlin (1984) and E. Gross and J. Meienhofer, Eds., The Peptides: Analysis, Synthesis, Biology, supra, Vol. 1, for classical solution synthesis.

The aqueous suspension may consist essentially of water and adjuvant having an antigen adsorbed thereon, or further additives may be included in the suspension. Any additives may be employed provided that they are substantially non-toxic and pharmacologically inert. The spray freeze-drying process has been found to be effective when applied to suspensions comprising a wide range of different additives and, as yet, the process of the invention, and therefore the powders of the invention, have been found to be entirely formulation independent.

Typically, the aqueous suspension comprises suitable excipients, along with protectants, solvents, salts, surfactants, buffering agents and the like. Suitable excipients can include free-flowing particulate solids that do not thicken or polymerize upon contact with water, which are innocuous when administered to an individual, and do not significantly interact with the pharmaceutical agent in a manner that alters its pharmaceutical activity. Examples of normally employed excipients include, but are not limited to, monosaccharides such as glucose, xylose, galactose, fructose, D-mannose or sorbose, disaccharides such as lactose, maltose, saccharose, trehalose or sucrose, sugar alcohols such as mannitol, sorbitol, xylitol, glycerol, erythritol or arabitol, polymers such as dextran, starch, cellulose or high molecular weight polyethylene glycols (PEG), amino acids or their salts, such as glycine, alanine, glutamine, arginine, lysine or histidine or their salts with alkali or alkaline earth metals such as a sodium, potassium or magnesium salts, or sodium or calcium phosphates, calcium carbonate, calcium sulfite, sodium citrate, citric acid, tartaric acid, and combinations thereof. Suitable solvents include, but are not limited to, methylene chloride, acetone, methanol, ethanol, isopropanol and water. Typically, water is used as the solvent. Generally pharmaceutically acceptable salts having molarities ranging from about 1 mM to 2M can be used. Pharmaceutically acceptable salts include, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients, vehicles and auxiliary substances is available in REMINGTON'S PHARMACEUTICAL SCIENCES (Mack Pub. Co., N.J. 1991), incorporated herein by reference.

Preferred excipients for use in the aqueous suspension include saccharides, amino acids or salts thereof and polymers. Typically, the suspension contains one or more saccharides, such as a combination of mannitol and trehalose. Saccharides are typically present in an amount of from 0.5 to 30% by weight. An amino salt, such as arginine glutamate or aspartate in an amount of from 0.1 to 30% by weight, and/or a polymer, such as dextran, in an amount of from 0 to 30% may also be included, typically in an amount of from 0 to 30% by weight. Typical excipient combinations include one or more saccharides and a polymer and include substantially no amino salt. The total amount of excipients present in the aqueous suspension is typically from 0 to 50%, more preferably from 10 to 30%.

The particles of the invention are formed by first suspending the adjuvant having an antigen adsorbed therein, and any required additives, in water. The aqueous suspension is then spray freeze-dried. Any known technique in the art (for example the methods described by Mumenthaler et al, Int. J. Pharmaceutics (1991) 72, pages 97-110 and Maa et al, Phar. Res. (1999) Vol. 16, page 249) may be used to carry out the spray freeze-drying step. A typical spray freeze-drying technique involves atomising the aqueous suspension into stirred liquid nitrogen. The liquid nitrogen containing frozen particles is then held at reduced temperature, for example from −60° C. to −20° C., followed by vacuum drying preferably under a pressure of from 20 to 500 mT (2.666 to 66.65 Pa), and at reduced temperature such as from −50° C. to 0° C. Drying is typically carried out in two stages, primary drying and secondary drying. Primary drying time typically ranges from 4 to 24 hours and secondary drying time typically ranges from 6 to 24 hours. The temperature may be gradually increased, whilst still under reduced pressure until room temperature is reached.

This technique involves the rapid freezing of the aqueous suspension into droplets. The drying step then removes the ice by sublimation without the need for high air temperatures. The powder may be collected by any known technique. The precise spray freeze-drying conditions used may be selected according to the desired properties of the particles to be produced. Thus, the temperatures, pressures and other conditions may be varied as desired.

The powders of the invention are generally free-flowing. The powders contain very little or no agglomerated adjuvant salt and are therefore capable of forming a gel on resuspension in water. Typically, substantially no precipitate forms upon resuspension. After a powder has been added to distilled water (1:500 by weight) and shaken for three minutes, a gel-like suspension without any precipitate is typically obtained. No precipitates settling out are observed after 3 hours. No precipitates may form after standing overnight, for example for 12 hours.

The presence of a precipitate, and the degree of agglomeration of the reconstituted gel formulation, is typically assessed by the ability of the reconstituted formulation to diffract a beam of light. The degree of agglomeration can also be quantitatively assessed by standard light microscopy and/or sedimentation. Another suitable test for particle agglomeration can be to determine particle size before and after reconstitution using any of a number of standard particle size determination techniques, e.g. laser-based or light obscuration.

The particles of the invention have a size appropriate for high-velocity transdermal delivery to a subject, typically across the stratum corneum or a transmucosal membrane. The mass mean aerodynamic diameter (MMAD) of the particles is from about 0.1 to 250 μm. The MMAD may be from 5 to 100 μm or from 10 to 100 μm, preferably from 10 to 70 μm or from 20 to 70 μm. Generally, less than 10% by weight of the particles have a diameter which is at least 5 μm more than the MMAD or at least 5 μm less than the MMAD. Preferably, no more than 5% by weight of the particles have a diameter which is greater than the MMAD by 5 μm or more. Also preferably, no more than 5% by weight of the particles have a diameter which is smaller than the MMAD by 5 μm or more.

The particles have an envelope density of from 0.1 to 25 g/cm³, preferably from 0.8 to 1.5 g/cm³. While the shape of the individual particles may vary when viewed under a microscope, the particles are preferably substantially spherical. The average ratio of the major axis:minor axis is typically from 3:1 to 1:1, for example from 2:1 to 1:1.

The individual particles of a powder have a substantially spherical aerodynamic shape with a substantially uniform, nonporous surface. The particles will also have a particle penetration energy suitable for transdermal delivery from a needleless syringe device.

A detailed description of needleless syringe devices useful in this invention is found in the prior art, as discussed herein. These devices are referred to as needleless syringe devices and representative of these devices are the dermal PowderJect® needleless syringe de-vice and the oral PowderJect® needleless syringe device (PowderJect Technologies Limited, Oxford, UK). By using these devices, an effective amount of the powder of the invention is delivered to the subject. An effective amount is that amount needed to deliver sufficient of the desired antigen to achieve vaccination. This amount will vary with the nature of the antigen and can be readily determined through clinical testing based on known activities of the antigen being delivered. The “Physicians Desk Reference” and “Goodman and Gilman's The Phamacological Basis of Therapeutics” are useful for the purpose of determined the amount needed.

Needleless syringe devices for delivering particles were first described in commonly owned U.S. Pat. No. 5,630,796 to Bellhouse et al, incorporated herein by reference. Although a number of specific device configurations are now available, such devices are typically provided as a pen-shaped instrument containing, in linear order moving from top to bottom, a gas cylinder, a particle cassette or package, and a supersonic nozzle with an associated silencer medium. An appropriate powder (in the present case, a spray-dried or spray freeze-dried powder of the invention) is provided within a suitable container, e.g., a cassette formed by two rupturable polymer membranes that are heat-sealed to a washer-shaped spacer to form a self-contained sealed unit. Membrane materials can be selected to achieve a specific mode of opening and burst pressure that dictate the conditions at which the supersonic flow is initiated. In operation, the device is actuated to release the compressed gas from the cylinder into an expansion chamber within the device. The released gas contacts the particle cassette and, when sufficient pressure is built up, suddenly breaches the cassette membranes sweeping the particles into the supersonic nozzle for subsequent delivery. The nozzle is designed to achieve a specific gas velocity and flow pattern to deliver a quantity of particles to a target surface of predefined area. The silencer is used to attenuate the noise produced by the membrane rupture.

A second needleless syringe device for delivering particles is described in commonly owned International Publication No. WO 96/20022. This delivery system also uses the energy of a compressed gas source to accelerate and deliver powdered compositions; however, it is distinguished from the system of U.S. Pat. No. 5,630,796 in its use of a shock wave instead of gas flow to accelerate the particles. More particularly, an instantaneous pressure rise provided by a shock wave generated behind a flexible dome strikes the back of the dome, causing a sudden eversion of the flexible dome in the direction of a target surface. This sudden eversion catapults a powdered composition (which is located on the outside of the dome) at a sufficient velocity, thus momentum, to penetrate target tissue, e.g., oral mucosal tissue. The powdered composition is released at the point of full dome eversion. The dome also serves to completely contain the high-pressure gas flow, which therefore does not come into contact with the tissue. Because the gas is not released during this delivery operation, the system is inherently quiet. This design can be used in other enclosed or otherwise sensitive applications for example, to deliver particles to minimally invasive surgical sites.

In yet a further aspect of the invention, single unit dosages or multidose containers, in which a powder of the invention may be packaged prior to use, can comprise a hermetically sealed container enclosing a suitable amount of the powder that makes up a suitable dose. The powder can be packaged as a sterile formulation, and the hermetically sealed container can thus be designed to preserve sterility of the formulation until use. If desired, the containers can be adapted for direct use in the above-referenced needleless syringe systems.

Powders of the present invention can thus be packaged in individual unit dosages for delivery via a needleless syringe. As used herein, a “unit dosage” intends a dosage receptacle containing a therapeutically effective amount of a powder of the invention. The dosage receptacle typically fits within a needleless syringe device to allow for transdermal delivery from the device. Such receptacles can be capsules, foil pouches, sachets, cassettes or the like.

The container in which the powder is packaged can further be labeled to identify the composition and provide relevant dosage information. In addition, the container can be labeled with a notice in the form prescribed by a governmental agency, for example the Food and Drug Administration, wherein the notice indicates approval by the agency under Federal law of the manufacture, use or sale of the powder contained therein for human administration.

The actual distance which the delivered particles will penetrate a target surface depends upon particle size (e.g., the nominal particle diameter assuming a roughly spherical particle geometry), particle density, the initial velocity at which the particle impacts the surface, and the density and kinematic viscosity of the targeted skin tissue. In this regard, optimal particle densities for use in needleless injection generally range between about 0.1 and 25 g/cm³ such as between about 0.8 and 1.7 g/cm³, preferably between about 0.9 and 1.5 g/cm³. Injection velocities generally range between about 100 and 3,000 m/sec. With appropriate gas pressure, particles having an average diameter of 10-70 μm can be accelerated through the nozzle at velocities approaching the supersonic speeds of a driving gas flow.

If desired, the needleless syringe systems can be provided in a preloaded condition containing a suitable dosage of the powder of the invention. The loaded syringe can be packaged in a hermetically sealed container, which may further be labeled as described above.

A number of novel test methods have been developed, or established test methods modified, in order to characterize performance of a needleless syringe device. These tests range from characterization of the powdered composition, assessment of the gas flow and particle acceleration, impact on artificial or biological targets, and measures of complete system performance. One, several or all of the following tests can thus be employed to assess the physical and functional suitability of the powder of the invention for use in a needleless syringe system.

Assessment of Effect on Artificial Film Targets

A functional test that measures many aspects of powder injection systems simultaneously has been designated as the “metallized film” or “penetration energy” (PE) test. It is based upon the quantitative assessment of the damage that particles can do to a precision thin metal layer supported by a plastic film substrate. Damage correlates to the kinetic energy and certain other characteristics of the particles. The higher the response from the test (i.e., the higher the film damage/disruption) the more energy the device has imparted to the particles. Either electrical resistance change measurement or imaging densitometry, in reflectance or transmission mode, provide a reliable method to assess device or formulation performance in a controllable and reproducible test.

The film test-bed has been shown to be sensitive to particle delivery variations of all major device parameters including pressure, dose, particle size distribution and material, etc. and to be insensitive to the gas. Aluminum of about 350 Angstrom thickness on a 125 μm polyester support is currently used to test devices operated at up to 60 bar.

Assessment of Impact Effect on Engineering Foam Targets

Another means of assessing particle performance when delivered via a needleless syringe device is to gauge the effect of impact on a rigid polymethylimide foam (Rohacell 5 IIG, density 52 kg/m³, Rohm Tech Inc., Malden, Mass.). The experimental set-up for this test is similar to that used in the metallized film test. The depth of penetration is measured using precision calipers. For each experiment a processed mannitol standard is run as comparison and all other parameters such as device pressure, particle size range, etc., are held constant. Data also show this method to be sensitive to differences in particle size and pressure. Processed mannitol standard as an excipient for drugs has been proven to deliver systemic concentrations in preclinical experiments, so the relative performance measure in the foam penetration test has a practical in vivo foundation. Promising powders can be expected to show equivalent or better penetration to mannitol for anticipation of adequate performance in preclinical or clinical studies. This simple, rapid test has value as a relative method of evaluation of powders and is not intended to be considered in isolation.

Particle Attrition Test

A further indicator of particle performance is to test the ability of various candidate compositions to withstand the forces associated with high-velocity particle injection techniques, that is, the forces from contacting particles at rest with a sudden, high velocity gas flow, the forces resulting from particle-to-particle impact as the powder travels through the needleless syringe, and the forces resulting from particle-to-device collisions also as the powder travels through the device. Accordingly, a simple particle attrition test has been devised which measures the change in particle size distribution between the initial composition, and the composition after having been delivered from a needleless syringe device.

The test is conducted by loading a particle composition into a needleless syringe as described above, and then discharging the device into a flask containing a carrier fluid in which the particular composition is not soluble (e.g., mineral oil, silicone oil, etc.). The carrier fluid is then collected, and particle size distribution in both the initial composition and the discharged composition is calculated using a suitable particle sizing apparatus, e.g., an AccuSizer® model 780 Optical Particle Sizer. Compositions that demonstrate less than about 50%, more preferably less than about 20% reduction in mass mean diameter (as determined by the AccuSizer apparatus) after device actuation are deemed suitable for use in the needleless syringe systems described herein.

Delivery to Human Skin In Vitro and Transepidermal Water Loss

For a powder performance test that more closely parallels eventual practical use, candidate powder compositions can be injected into dermatomed, full thickness human abdomen skin samples. Replicate skin samples after injection can be placed on modified Franz diffusion cells containing 32° C. water, physiologic saline or buffer. Additives such as surfactants may be used to prevent binding to diffusion cell components. Two kinds of measurements can be made to assess performance of the formulation in the skin.

To measure physical effects, i.e. the effect of particle injection on the barrier function of skin, the transepidermal water loss (TEWL) can be measured. Measurement is performed at equilibrium (about 1 hour) using a Tewameter TM 210® (Courage & Khazaka, Koln, Ger) placed on the top of the diffusion cell cap that acts like a ˜12 mm chimney. Larger particles and higher injection pressures generate proportionally higher TEWL values in vitro and this has been shown to correlate with results in vivo. Upon particle injection in vitro TEWL values increased from about 7 to about 27 (g/m²h) depending on particle size and helium gas pressure. Helium injection without powder has no effect. In vivo, the skin barrier properties return rapidly to normal as indicated by the TEWL returning to pretreatment values in about 1 hour for most powder sizes. For the largest particles, 53-75 μm, skin samples show 50% recovery in an hour and full recovery by 24 hours.

Delivery to Human Skin in vitro and Drug Diffusion Rate

To measure the formulation performance in vitro, the antigen component(s) of candidate powders can be collected by complete or aliquot replacement of the Franz cell receiver solution at predetermined time intervals for chemical assay using HPLC or other suitable analytical technique. Concentration data can be used to generate a delivery profile and calculate a steady state permeation rate. This technique can be used to screen formulations for early indication of antigen binding to skin, antigen dissolution, efficiency of particle penetration of stratum corneum, etc., prior to in vivo studies.

These and other qualitative and quantitative tests can be used to assess the physical and functional suitability of the present powders for use in a high-velocity particle injection device. It is preferred, though not required, that the particles of a powder have the following characteristics: a substantially spherical shape (e.g. an aspect ratio as close as possible to 1); a smooth surface; a suitable active loading content; less than 20% reduction in particle size using the particle attrition test; an envelope density as close as possible to the true density of the constituents (e.g. greater than about 0.8 g/ml); and a MMAD of about 20 to 70 μm with a narrow particle size distribution. The compositions are typically free-flowing (e.g. free-flowing after 8 hours storage at 50% relative humidity and after 24 hours storage at 40% relative humidity). All of these criteria can be assessed using the above-described methods, and are further detailed in the following publications, incorporated herein by reference. Etzler et al (1995) Part. Part. Syst. Charact. 12:217; Ghadiri, et al (1992) IFPRI Final Report, FRR 16-03 University of Surrey, UK; Bellhouse et al (1997) “Needleless delivery of drugs in dry powder form, using shock waves and supersonic gas flow,” Plenary Lecture 6, 21^{st} International Symposium on Shock Waves, Australia; and Kwon et al (1998) Pharm. Sci. suppl. 1 (1), 103.

A powder of the invention may alternatively be used to vaccinate a subject via other routes. For this purpose, the powder may be combined with a suitable carrier or diluent such as Water for Injections or physiologically saline. The resulting vaccine composition is typically administered by injection, for example subcutaneously or intramuscularly.

Whichever route of administration is selected, an effective amount of antigen is delivered to the subject being vaccinated. Generally from 50 ng to 1 mg and more preferably from 1 μg to about 50 μg of antigen will be useful in generating an immune response. The exact amount necessary will vary depending on the age and general condition of the subject to be treated, the particular antigen or antigens selected, the site of administration and other factors. An appropriate effective amount can be readily determined by one of skill in the art.

Dosage treatment may be a single dose schedule or a multiple dose schedule. A multiple dose schedule is one in which a primary course of vaccination may be with 1-10 separate doses, followed by other doses given at subsequent time intervals, chosen to maintain and/or reinforce the immune response, for example at 1-4 months for second dose and, if needed, a subsequent dose(s) after several months. The dosage regimen will also, at least in part, be determined by the need of the subject and be dependent on the judgement of the practitioner. Vaccination will of course generally be effected prior to primary infection with the pathogen against which protection is desired.

C. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### REFERENCE EXAMPLE 1

A spray-dried immediate-release vaccine preparation was obtained according to the procedure described in U.S. Pat. No. 5,902,565. A formulation containing 5% by weight mannitol and 5% by weight aluminum phosphate (Adju-Phos) was spray dried using a bench-top spray dryer (Buchi 190). The spray-drying conditions were: inlet temperature=130° C.; outlet temperature =70° C., liquid feed rate=3 ml/min; atomizing airflow rate=500 l/hr; and a full scale of drying air. The free-flowing powder that was obtained had a particle size of about 10 μm. The powder was reconstituted in distilled water (1:500 by weight). The solution failed to form a gel with the suspended particles setting in 15 minutes. By optical microscopy, the particles after reconstitution maintained their shape and size, suggesting that the alum remained coagulated and did not disintegrate.

### EXAMPLE 1

The following formulations were prepared by mixing the components listed in the Table below in 15 ml of distilled water:

| Formulation | Aluminum Salt | | Mannitol | Glycine | Dextran |
|---|---|---|---|---|---|
| 1 (comparison) | 14.5 | g of Alhydrogel¹⁾ | 322 mg | 131 mg | 17.5 mg |
| 2 (invention) | 2.5 | g of Alhydrogel¹⁾ | 693 mg | 130 mg | 18 mg |
| 3 (comparison) | 15 | g of Adju-Phos²⁾ | 438 mg | 173 mg | 16.9 mg |
| 4 (comparison) | 7.7 | g of Adju-Phos²⁾ | 882 mg | 172 mg | 16.2 mg |

¹⁾Alhydrogel: 3% by weight aluminum hydroxide
²⁾Adju-Phos: 2% by weight aluminum phosphate

These formulations were spray dried using a Buchi 190 Mini-Spin Drier operating under the following conditions: air inlet temperature =130° C.; air outlet temperature=70° C.; Q liquid feed: setting 5; and Q atomising air: 500 l/hr. Drying air was set at the full scale. Free-flowing powders were obtained. Yields were as follows:

| Formulation | Powder yield (g) | % Yield | MMAD |
|---|---|---|---|
| 1 | 0.52 | 68.4 | 8-10 μm |
| 2 | 0.48 | 53.9 | 8-10 μm |
| 3 | 0.91 | 74.1 | 8-10 μm |
| 4 | 0.38 | 31.0 | 8-10 μm |

The composition of the powders obtained in relation to the solids content of the suspension subjected to spray drying was as follows:

| | | Manni- | Gly- | Dex- | Total |
|---|---|---|---|---|---|
| | Al(OH)₃ | tol | cine | tran | Solid |
| Formulation 1 | | | | | |
| Solid content in suspension | 2.9 | 2.1 | 0.9 | 0.1 | 6 |
| for spray drying (%) | | | | | |
| Powder content | 48.3% | 35.0% | 15.0% | 1.7% | |
| Formulation 2 | | | | | |
| Solid content in suspension | 0.5 | 4.6 | 0.9 | 0.1 | 6.1 |
| for spray drying (%) | | | | | |
| Powder content | 8.2% | 75.4% | 14.8% | 1.6% | |
| Formulation 3 | | | | | |
| Solid content in suspension | 4 | 2.9 | 1.2 | 0.1 | 8.2 |
| for spray drying (%) | | | | | |
| Powder content | 48.8% | 35.4% | 14.6% | 1.2% | |
| Formulation 4 | | | | | |
| Solid content in suspension | 1 | 5.9 | 1.1 | 0.1 | 8.1 |
| for spray drying (%) | | | | | |
| Powder content | 12.3% | 72.8% | 13.6% | 1.2% | |

The spray dried powders were resuspended in distilled water. Specifically, each powder was added to distilled water (1:500 by weight) and shaken for 3 minutes. The resulting suspensions were examined for aggregation. Only Formulation 2 according to the invention formed a gel-like suspension without precipitate. The results are shown below:
Formulation 1: 32.59 mg of spray-dried powder was added to 1 ml of distilled water. A white precipitate formed after the resulting suspension has been allowed to stand overnight.
Formulation 2: 37.1 mg of spray-dried powder was added to 1 ml of distilled water. An off-white, grey, gel-like suspension formed. No precipitate was observed after the suspension had been allowed to stand overnight.
Formulation 3: 44.34 mg of spray-dried powder was added to 1 ml of distilled water A white precipitate formed after the resulting suspension had been allowed to stand overnight.
Formulation 4: 29.4 mg of spray-dried powder was added to 1 ml of distilled water. A white precipitate formed after the resulting suspension had been allowed to stand overnight.

### EXAMPLE 2

Two vaccine formulations were prepared as follows:

Formulation A:

A concentrated alum-HBsAg suspension was prepared by first washing an alum-adsorbed HBsAg vaccine obtained from Rhein Americana S. A. containing 20 μg of HBsAg (approximately 1 human dose) adsorbed on 500 μg of alum (approximately 1500 μg of aluminum hydroxide) with distilled, deionised water to remove buffer salt. Alum gel was allowed to settle overnight in a 250-mL Nalgene narrow-mouth square polycarbonate bottle at 2-8° C. The supernatant (150 mL) was removed and the same volume of water was added to the precipitates and mixed. This procedure was repeated for a second time.

100 g of the washed alum-HBsAg formulation was weighed in a Nalgene square bottle and allowed to settle overnight at 2-8° C. After 90 mL of supernatant was removed, the remaining suspension was transferred to a 50 mL polypropylene centrifuge tube and centrifuged at 200 rpm for 4 minutes using a bench-top centrifuge (Allegra 6R, Beckman). The supernatant was further removed to obtain 3.369 g of concentrated alum-HBsAg suspension. This suspension was then mixed with 315.24 mg mannitol, 81.73 mg glycine, 101.91 mg dextran and placebo alum gel (Al₂O₃ at 2%) to achieve a liquid alum-HBsAg formulation having an alum concentration of 3%.

Formulation B:

An alum-HBsAg suspension was washed in accordance with the method described for formulation A. 20.79 g of the suspension was weighed in a 50 mL centrifuge tube and allowed to settle overnight at 2-8° C. After 17 mL of supernatant was removed, the remaining concentrated suspension (3.572 g) was mixed with 113.06 mg mannitol, 47.31 mg glycine and 23.22 mg dextran to produce a liquid formulation having an alum concentration of 0.6%.

The two formulations were dried using the techniques set out in Table 1 below:

TABLE 1
Drying techniques

| Powder | Formulation | Drying technique |
|---|---|---|
| 1 (comparison) | A | Freeze-drying |
| 2 (invention) | A | Spray freeze-drying |
| 3 (invention) | B | Spray freeze-drying |
| 4 (comparison) | A | Freeze-drying followed by C/G/S |
| | | (using <20 μm fraction) |
| 5 (comparison) | A | Freeze-drying followed by C/G/S |
| | | (using 38-45 μm fraction) |
| 6 (comparison) | A | Freeze-drying followed by C/G/S |
| | | (using 53-75 μm fraction) |

Freeze Drying:

A Dura-Stop freeze dryer (FTS System, Stone Ridge, N.Y.) was used to freeze dry the alum-adsorbed HBsAg formulation based on the freeze-drying cycle in Table 2.

TABLE 2
Freeze-drying cycle

| Stage/Cycle | Conditions |
|---|---|
| Freezing | pre-cool shelf temperature (ST) = 0° C. |
| | ramp at 1.0° C./min to ST = −55° C., |
| | hold for 15 min |
| | wait for product temp (PT) = −48° C., |
| | hold for 120 min |
| Primary | condenser/vacuum (C/V) switched “on” |
| Drying | when condenser temp. reaches −40° C., |
| | vacuum pump turned on |
| | wait for chamber vacuum to reach 150 mT (20.0 Pa) |
| | wait for foreline vacuum to reach 100 mT (13.3 Pa) |
| | ramp at 1.0° C./min to ST = −25° C., |
| | hold for 18 hours |
| Secondary | ramp at 1.0° C./min to ST = 10° C., |
| Drying | hold for 4 hours |
| | ramp at 1.0° C./min to ST = 20° C., |
| | hold for 11 hours |

A vacuum of 100 mT (13.3 Pa) was maintained throughout primary and secondary drying.

Spray-Freeze-Drying:

Each suspension solution was sprayed into liquid nitrogen stirred in a stainless steel pain using an ultrasonic atomizer (Sono Tek Corporation, Milton, N.Y.) with a nozzle frequency of 60 kHz. Sonic energy for atomization was set at 5.0 watts. Liquid feed was delivered by a MasterFlex C/L peristaltic pump at 1.5 mL/min. The pan containing frozen particles in liquid nitrogen was loaded into the Dura-lyophilizer pre-cooled to −50° C. and freeze-dried based on the condition of Table 3.

TABLE 3
Freeze-drying cycle

| Stage/Cycle | Conditions |
|---|---|
| Freezing | pre-cool shelf temperature (ST) = −50° C. |
| | ramp at 1.0° C./min to ST = −55° C., |
| | hold for 15 min |
| | wait for product temp (PT) = −48° C., |
| | hold for 120 min |
| Primary | condenser/vacuum (C/V) switched “on” |
| Drying | when condenser temp. reaches −40° C., |
| | vacuum pump turned on |
| | wait for chamber vacuum to reach 150 mT (20.0 Pa) |
| | wait for foreline vacuum to reach 100 mT (13.3 Pa) |
| | ramp at 1.0° C./min to ST = −25° C., |
| | hold for 18 hours |
| Secondary | ramp at 1.0° C./min to ST = 20° C., |
| Drying | hold for 9 hours |

A vacuum of 200 mT (16.6 Pa) was maintained throughout primary and secondary drying.

Compress/Grind/Sieve:

The lyophilized material was rendered into particulate form using a compress, grind and sieve (“C/G/S”) technique. More particularly, the lyophilized material was compressed in a stainless steel dye of 13-mm in diameter (Carver Press, Wabash, Ind.) at a pressure of 12,000 psi for 5-10 minutes. The compressed discs were ground manually using a mortar and pestle. The ground powder was manually sieved through a stack of sieves (3-in diameter) into three size fractions, 53-75 μm, 38-53 μm, and 20-38 μm.

Experiment 1: Effect of Drying Process on the Extent of Coagulation

Powders 1 to 3 were reconstituted in water at a ratio of 1:500 w/w and examined using optical microscopy in accordance with standard techniques. Visual analysis of the particles was performed using an optical microscope (Model DMR, Leica, Germany) with 10×-eyepeice lens and 5×-objective lens. The system was equipped with a Polaroid camera system for image output. Optical microscopy provides a qualitative analysis of the degree of alum coagulation. In this experiment, powder 1 produced very large aggregates on reconstitution, whereas powder 2 coagulated only slightly. Powder 3 produced almost no aggregates at all.

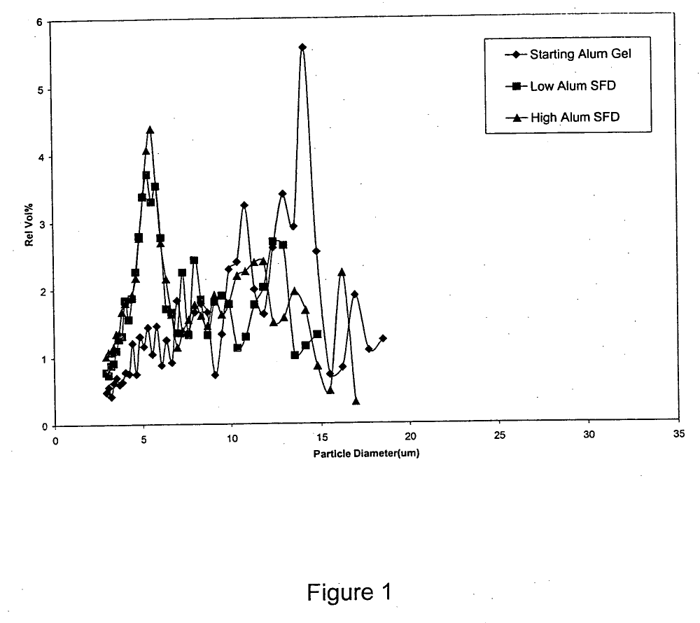

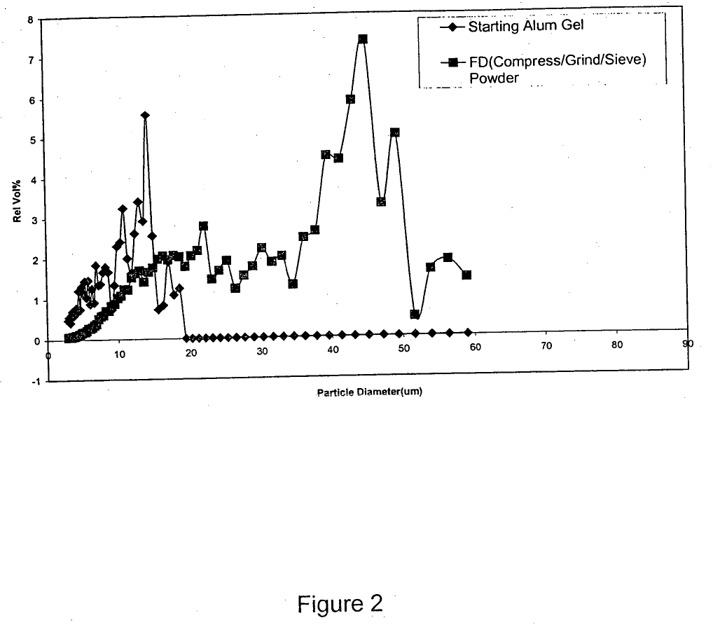

The particle size of the reconstituted powders was also measured quantitatively. The reconstituted powder sample was vortexed/sonicated to make a homogeneous suspension. The suspension was then added to the glass container of a particle size analyzer (AccuSizer 780, Particle Sizing Systems, Santa Barbara, Calif.) for particle size distribution measurement. The results of the measurements carried out on powders 2 and 3 both before and after spray freeze-drying are shown in FIG. 1. Similar comparative results for powder 1 showing particle size before and after freeze-drying are shown in FIG. 2. These results illustrate the similar particle size distribution of powders 2 and 3 before and after drying, demonstrating that little or no alum coagulation occurred during freeze-drying. In contrast, the particle size of powder 1 increases significantly after freeze-drying, indicating that significant alum coagulation has occurred.

Experiment 2: Effect of Coagulation on the Stability of Alum Containing Hepatitis B Vaccine

A study was carried out to assess the effect of alum coagulation on the immunogenicity of alum-absorbed hepatitis B vaccine. As stated earlier, severe coagulation occurred when hepatitis B vaccine (containing alum) was dried by the freeze-drying process, whereas spray-freeze-drying of hepatitis B vaccine did not cause coagulation. In this mouse experiment, the immunogenicity of freeze-dried and spray-freeze-dried hepatitis B vaccines were compared. Further, the immunogenicity of unsieved free-dried vaccine and various sieved fractions (<20, 38-45, 53-75 μm in diameter) were compared to determine which size fraction was more immunogenic. The experimental design is shown in Table 4.

TABLE 4
Experimental design of the mouse immunogenicity study

| | Formu- | | Particle | | Injection route |
|---|---|---|---|---|---|
| Group | lation * | Drying Technique | size | | (reconstituted) |
| 1 | A | freeze-drying | unsieved | | intraperitoneal |
| 2 | A | freeze-drying | <20 | μm | intraperitoneal |
| 3 | A | freeze-drying | 38-45 | μm | intraperitoneal |
| 4 | A | freeze-drying | 53-75 | μm | intraperitoneal |
| 5 | A | Spray-freeze-drying | 10-75 | μm | intraperitoneal |
| 6 | B | Spray-freeze-drying | 10-75 | μm | intraperitoneal |

| | | | | |
|---|---|---|---|---|
| 7 | Not | Liquid alumn vaccine | — | intraperitoneal |
| | treated | used | | |

* Details of the formulation A and B are described above

Powders were reconstituted with distilled water and used to immunize Balb/C mice (female, 8 per group, 5-7 weeks old at the beginning of the study). Reconstituted vaccines were administered by intraperitoneal injection using a 23⅕ needle. Each injection administered 200 μl of solution containing 2 μg of hepatitis B surface antigen absorbed on alum. Control mice were immunized with untreated liquid hepatitis B vaccine. Following a prime (day 0) and a boost immunisation (day 28), immune responses to the hepatitis B vaccine were determined with serum collected on day 42 in an ELISA. The antibody titers were determined by comparing to reference a serum.

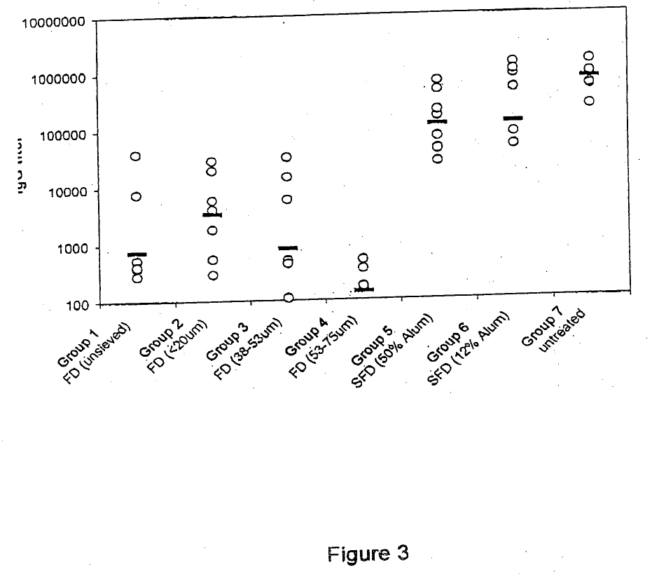

The results of these trials, as set out in FIG. 3, clearly indicated that the alum coagulation caused by freeze-drying resulted in a decrease and even loss of immunogencity of the hepatitis B vaccine. Compared to the untreated liquid vaccine, freeze-dried hepatitis B vaccine (group 1) had diminishing immunogenicity. The immunogenicity of the freeze-dried particle had an adverse correlation with the size of the particles (groups 2, 3 and 4). The larger particle fractions were less immunogenic than the smaller particle size fraction. This clearly indicated that large size particles associated with coagulation had lost its vaccine potency. The spray-freeze dried hepatitis B vaccine maintained its immunogenicity (groups 5 and 6) when compared with the untreated vaccine. The amount of alum in the total dry mass (50% or 12%) did not affect the potency of the dry powder. Neither of the spray-freeze-dried powders had a coagulation problem. This is significant that the spray-freeze-drying formulation preserves the potency of alum salt adjuvant at a very high concentrations (3% by weight).

Taken together all these data, it can be concluded that alum coagulation is associated with the potency loss of alum vaccine when freeze-dried. It is believed that the large sizes of coagulated particles, which may fail to solubilize in vivo, can not be processed by the cells of the immune system and, thus, have no potency. More importantly, the process of the invention can prepare stable dry powders with alum containing vaccine without causing coagulation. It is believed that the quick freezing in the liquid nitrogen employed in the spray-freeze-drying process is critical for preventing the coagulation, thus preserving the vaccine potency.

Experiment 3: Effect of Excipient and Drying Processes on the Stability of Spray-Freeze-Dried Hepatitis B Vaccine

In this study, the effect of excipients and a variant spray-freeze-drying process on the stability of alum vaccines was evaluated. Hepatitis B surface antigen (HBsAg) absorbed on alum hydroxide was used as a model antigen. In addition, the immunogenicity of spray-freeze-dried powders was evaluated in mice following two different routes of immunisation, intramuscular injection using a needle and epidermal powder immunisation using a needleless powder delivery device. The excipients for the spray-freeze-dried formulations are shown in Table 5. In this case, the spray-freeze-dried formulations used the combination of two sugars and one polymer. There was no amino acid/salt involved. The conditions for spray-freeze-drying are the same as that shown in Table 3. However, compress/grind/sieve step was not used. The particle size distribution of the spray-freeze-dried powders is also indicated in Table 5.

TABLE 5
Composition of spray-freeze-drying formulations

| | | | | | Particle size, | | |
|---|---|---|---|---|---|---|---|
| Formu- | | | | | μm (Aerosizer) | | |
| lation | Vaccine | | Excipient | Process | Dv10 | Dv50 | Dv95 |
| SFD-C | 2 | μg HBsAg/ | Trehalose/mannitol/ | Spray- | 23 | 38 | 57 |
| | 50 | μg Alum | PEG (3:4:3) | freeze-dry | | | |
| SFD-D | 2 | μg HBsAg/ | Trehalose/mannitol/37 | Spray- | 26 | 39 | 59 |
| | 50 | μg Alum | kD dextran (3:4:3) | freeze-dry | | | |
| SFD-E | 2 | μg HBsAg/ | Trehalose/mannitol/10 | Spray- | 24 | 36 | 56 |
| | 50 | μg Alum | kD dextran (3:4:3) | freeze-dry | | | |

The immunogenicity of spray-freeze-dried formulations was evaluated in a mouse study. Balb/C mice (female, 8 per group, 5-7 weeks old at the beginning of the study) were used. The study design is shown in Table 6. For intramuscular (IM) injection, powders were reconstituted with distilled water and administered by injection 200 μl of solution containing 2 μg of hepatitis B surface antigen absorbed on alum into the quadriceps muscle using a 23⅕ needle. For epidermal (EPI) powder immunisation, powders were administered to the shaved abdominal skin of mice using a re-chargeable powder delivery device. Control mice were immunised with untreated liquid hepatitis B vaccine by intramuscular injection. Following a prime (day 0) and a boost immunisation (day 28), immune responses to the hepatitis B vaccine were determined with serum collected on day 42 in an ELISA. The antibody titers were determined by comparing to reference a serum.

TABLE 6
Experimental design of the mouse immunogenicity study

| Group | Formulation | Reconstitution | Route |
|---|---|---|---|
| 1 | SFD-C | yes | IM |
| 2 | SFD-D | yes | IM |
| 3 | SFD-E | yes | IM |
| 4 | SFD-C | no | EPI |
| 5 | SFD-D | no | EPI |
| 6 | SFD-E | no | EPI |
| 7 | untreated | Not applicable | IM |

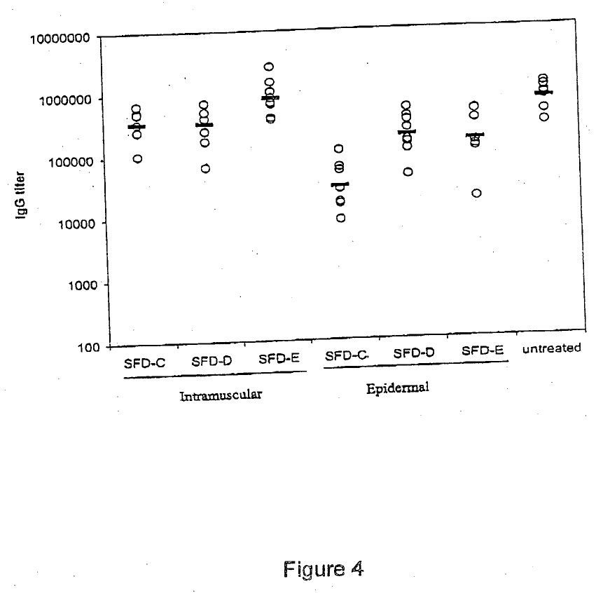

The results of this study, as shown in FIG. 4, clearly indicate that all three spray-freeze-dried hepatitis B vaccines are immunogenic in mice whether it is administered by the intramuscular route after reconstitution or by the epidermal route as powders. Different excipients were used in these formulations and there were no significant differences in the immunogenicity among these formulations. All three formulations had no coagulation problem when reconstituted in water (data not shown). This provides further evidence that the quick-freezing step in the spray-freeze-drying process is a critical step to stabilize the alum. Excipients may play a less important role. This study also demonstrated that spray-freeze-dried vaccines absorbed on alum can be useful for immunisation via different routes, e.g. intramuscularly injection when reconstituted or epidermal powder immunisation in a powder form.

Experiment 4: Immunogenicity of Spray-Freeze-Dried Diphtheria-Tetanus Toxoid Vaccine

To determine of spray-freeze-drying process can be used prepare stable powders with other alum-containing vaccine, spray-freeze dried powders using diphtheria-toxoid vaccine obtained from CSL Limited (Australia) were prepared. This bulk contained 5% w/v aluminium phosphate adsorbed with both diphtheria toxoid and tetanus toxoid at a concentration of 563 Lf/mL each. The spray freeze-dried diphtheria-tetanus-toxoid vaccine was prepared under the conditions as described in Table 3 and followed by compress/grind/sieve to generate particles with mean size of 20-38 μm and 38-53 μm in diameter. The formulation information is summarised in Table 7. These particles do not have coagulation problems when reconstituted in water and examined under optical microscopy (data not shown).

TABLE 7

| HBsAg-Alum | Trehalose | | | Total solid | |
|---|---|---|---|---|---|
| phosphate | dihydrate | Glycine | Dextran | content | |
| (mg) | (mg) | (mg) | (mg) | (%) | DT dose |
| 250 | 292.9 | 66.1 | 86.6 | 4 | 3 Lf/1-mg |
| | | | | | powder |

The immunogenicity of spray-freeze-dried diphtheria-tetanus-toxoid vaccine was determined in guinea pigs (Charles River). Guinea pigs (4/group) were vaccinated on days 0 and 28 by administering powders to the abdominal skin using a powder delivery device. Each animal received 0.5 mg powders containing 1.5 Lf diphtheria toxoid and 1.5 Lf tetanus toxoid absorbed on 250 μg of aluminum phosphate. Control animals were vaccinated with untreated vaccine by intramuscular injection using a 23½ needle. Serum antibody responses to diphtheria toxoid and tetanus toxoid were measured in an ELISA using sera collected on days 42.

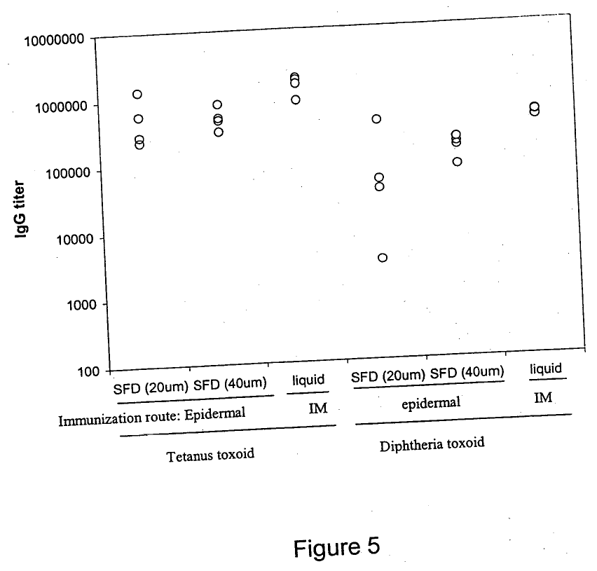

The results of the immunogenicity study are shown in FIG. 5. Epidermal powder immunisation with spray-freeze-dried diphtheria toxoid absorbed on alum elicited antibody responses to each of the vaccine components and the tiers are comparable to that elicited by intramuscular injection of untreated vaccine. The size of the spray-freeze-dried powders did not appear to affect the immunogenicity significantly since these powders did not have coagulation problem in vivo. The smaller particle fraction of the spray-freeze dried formulation appears to have elicited slightly lower antibody titers to the diphtheria toxoid than the larger size fraction. This may reflect the relatively lower delivery efficiency for the smaller size fraction. This study again demonstrated that spray-freeze-drying process preserves the potency of alum-containing vaccine the dry solid dosage form.

Accordingly, novel freeze spray-dried powder compositions and methods for producing these compositions have been described. Although preferred embodiments of the subject invention have been described, in some detail, it is understood that obvious variations can be made without departing from the spirit and the scope of the invention as defined by the appended claims.

1. A gel-forming free-flowing powder suitable for use as a vaccine, said powder being obtained by spray-drying or spray freeze-drying an aqueous suspension comprising:
(a) from 0.1 to 0.95% by weight of an aluminum salt or calcium salt adjuvant having an antigen adsorbed therein;
(b) from 0.5 to 6% by weight of a saccharide;
(c) from 0.1 to 2% by weight of an amino acid or salt thereof; and
(d) from 0.02 to 1% by weight of a colloidal substance.

2. A powder according to claim 1, wherein the adjuvant is aluminum hydroxide, aluminum phosphate, aluminum sulfate or calcium phosphate.

3. (canceled)

4. A powder according the claim 1, wherein the antigen is a bacterial or viral antigen.

5. (canceled)

6. A powder according to claim 1, wherein the saccharide is selected from the group consisting of glucose, xylose, galactose, fructose, D-mannose, sorbose, lactose, maltose, saccharose, trehalose, sucrose, mannitol, sorbitol, xylitole, glycerin, glycerol, erythritol and arabitol.

7. (canceled)

8. A powder according to claim 1, wherein the amino acid or salt thereof is selected from the group consisting of glycine, alanine, glutamine, arginine, lysine, histidine and monosodium glutamate.

9. (canceled)

10. A powder according to claim 1, wherein the colloidal substance is selected from the group consisting of dextran, maltodextran, gelatin, agarose and human serum albumin.

11. A powder according to claim 1, wherein the aqueous suspension comprises from 0.2 to 0.4% by weight of the adjuvant having antigen adsorbed thereon, from 2 to 4% by weight of the saccharide, from 0.75 to 1.25% by weight of the amino acid or salt thereof and from 0.07 to 0.3% by weight of the colloidal substance.

12. A powder according to claim 1, which comprises:
(i) from 7 to 50% by weight of the adjuvant having an antigen adsorbed therein,
(ii) from 30 to 80% by weight of the saccharide,
(iii) from 7 to 30% by weight of the amino acid or salt thereof, and
(iv) from 0.8 to 6% by weight of the colloidal substance.

13. A powder according to claim 1, having a mass mean aerodynamic diameter of from 10 to 100 μm and an envelope density of from 0.8 to 1.5 g/cm3.

14. A powder according to claim 1, which forms a gel-like suspension without any precipitate after having been added to distilled water (1:500 by weight) and shaken for 3 minutes.

15. A process for the preparation of a gel-forming free-flowing powder suitable for use as a vaccine, which process comprises the step of spray-drying or spray freeze-drying an aqueous suspension comprising:
from 0.1 to 0.95% by weight of an aluminum salt or calcium salt adjuvant having an antigen adsorbed therein;
from 0.5 to 6% by weight of a saccharide;
from 0.1 to 2% by weight of an amino acid or salt thereof; and
from 0.02 to 1% by weight of a colloidal substance.

16. A process according to claim 15 wherein the aqueous suspension comprises from 0.2 to 0.4% by weight of the adjuvant having antigen adsorbed thereon, from 2 to 4% by weight of the saccharide, from 0.75 to 1.25% by weight of the amino acid or salt thereof and from 0.07 to 0.3% by weight of the colloidal substance.

17. A process according to claim 15, wherein the resultant powder forms a gel-like suspension without any precipitate after having been added to distilled water (1:500 by weight) and shaken for 3 minutes.

18. A dosage receptacle for a needleless syringe, said receptacle containing an effective amount of a gel-forming free-flowing powder obtained by spray-drying or spray freeze-drying an aqueous suspension comprising:
(a) from 0.1 to 0.95% by weight of an aluminium salt or calcium salt adjuvant having an antigen adsorbed therein;
(b) from 0.5 to 6% by weight of a saccharide;
(c) from 0.1 to 2% by weight of an amino acid or salt thereof; and
(d) from 0.02 to 1% by weight of a colloidial substance.

19. A receptacle according to claim 18, wherein the receptacle is selected from the group consisting of capsules, foil pouches, sachets and cassettes.

20. A needleless syringe which is loaded with a gel-forming free-flowing powder obtained by spray-drying or spray freeze-drying an aqueous suspension comprising:
from 0.1 to 0.95% by weight of an aluminum salt or calcium salt adjuvant having an antigen adsorbed therein;
from 0.5 to 6% by weight of a saccharide;
from 0.1 to 2% by weight of an amino acid or salt thereof; and
from 0.02 to 1% by weight of a colloidal substance.

21. A vaccine composition comprising a pharmaceutically acceptable carrier or diluent and a gel-forming free-flowing powder obtained by spray-drying or spray freeze-drying an aqueous suspension comprising:
(a) from 0.1 to 0.95% by weight of an aluminum salt or calcium salt adjuvant having an antigen adsorbed therein;
(b) from 0.5 to 6% by weight of a saccharide;
(c) from 0.1 to 2% by weight of an amino acid or salt thereof; and
(d) from 0.02 to 1% by weight of a colloidal substance.

22. A method of vaccinating a subject, which method comprises the step of administering to the said subject an effective amount of a gel-forming free-flowing powder obtained by spray-drying or spray freeze-drying an aqueous suspension comprising:
(a) from 0.1 to 0.95% by weight of an aluminum salt or calcium salt adjuvant having an antigen adsorbed therein;
(b) from 0.5 to 6% by weight of a saccharide;
(c) from 0.1 to 2% by weight of an amino acid or salt thereof; and
(d) from 0.02 to 1% by weight of a colloidal substance.

23-25. (canceled)

26. A gel-forming free-flowing powder suitable for use as a vaccine, which powder comprises:
(i) from 5 to 60% by weight of an aluminum salt or calcium salt adjuvant having an antigen adsorbed thereon;
(ii) from 25 to 90% by weight of a saccharide;
(iii) from 4.5 to 40% by weight of an amino acid or salt thereof; and
(iv) from 0.5 to 10% by weight of a colloidal substance.

27. A powder according to claim 26, which comprises:
(i) from 7 to 50% by weight of the adjuvant having an antigen adsorbed therein,
(ii) from 30 to 80% by weight of the saccharide,
(iii) from 7 to 30% by weight of the amino acid or salt thereof, and
(iv) from 0.8 to 6% by weight of the colloidal substance.

28. A powder according to claim 26, which forms a gel-like suspension without any precipitate after having been added to distilled water (1:500 by weight) and shaken for 3 minutes.

29. A powder suitable for use as a vaccine, said powder being obtained by spray freeze-drying an aqueous suspension comprising an aluminum salt or calcium salt adjuvant having an antigen adsorbed therein.

30. A powder according to claim 29, wherein the adjuvant is aluminum hydroxide, aluminum phosphate, aluminum sulphate or calcium phosphate.

31. A powder according to claim 29, wherein the antigen is a bacterial or viral antigen.

32. A powder according to claim 29, wherein the aqueous suspension comprises less than 10% by weight of the adjuvant having antigen adsorbed thereon.

33. A powder according to claim 29, having a mass mean aerodynamic diameter of from 1 to 100 μm and an envelope density of from 0.8 to 1.5 g/cm3.

34. A powder according to claim 29, wherein the suspension further comprises an amorphous sugar, a crystalline sugar and optionally a polymer and/or an amino acid or a salt thereof.

35. A powder according to claim 29, which forms a gel-like suspension without any precipitate after having been added to distilled water (1:500 by weight) and shaken for 3 minutes.

36. A process for the preparation of a powder suitable for use as a vaccine, which process comprises the step of spray freeze-drying an aqueous suspension comprising an aluminum salt or calcium salt adjuvant having an antigen adsorbed therein.

37. A process according to claim 36, wherein the adjuvant is aluminum hydroxide, aluminum phosphate, aluminum sulphate or calcium phosphate.

38. A process according to claim 36, wherein the antigen is a bacterial or viral antigen.

39. A process according to 36, wherein the aqueous suspension comprises less than 10% by weight of the adjuvant having antigen adsorbed thereon.

40. A process according to claim 36, wherein the suspension further comprises an amorphous sugar, a crystalline sugar and optionally a polymer and/or an amino acid or a salt thereof.

41. A process according to claim 36, wherein the resultant spray freeze-dried powder forms a gel-like suspension without any precipitate after having been added to distilled water (1:500 by weight) and shaken for 3 minutes.

42. (canceled)

43. A dosage receptacle for a needleless syringe, said receptacle containing an effective amount of a powder obtained by spray freeze-drying an aqueous suspension comprising an aluminum salt or calcium salt adjuvant having an antigen adsorbed therein.

44. A receptacle according to claim 43, wherein the receptacle is selected from the group consisting of capsules, foil pouches, sachets and cassettes.

45. A needleless syringe which is loaded with a powder obtained by spray freeze-drying an aqueous suspension comprising an aluminum salt or calcium salt adjuvant having an antigen adsorbed therein.

46. A vaccine composition comprising a pharmaceutically acceptable carrier or diluent and a powder obtained by spray freeze-drying an aqueous suspension comprising an aluminum salt or calcium salt adjuvant having an antigen adsorbed therein.

47. A method of vaccinating a subject, which method comprises administering to the said subject an effective amount of a powder obtained by spray freeze-drying an aqueous suspension comprising an aluminum salt or calcium salt adjuvant having an antigen adsorbed therein.

48-50. (canceled)
